(19) United States
(12) Patent Application Publication
(10) Pub. No.: US 2021/0251832 A1
(43) Pub. Date: Aug. 19, 2021

(54) DEVICE FOR PREVENTING PRESSURE ULCERS

(71) Applicant: BETA MEDTECH PTE. LTD., Singapore (SG)

(72) Inventor: Edison BELLARMIN, Singapore (SG)

(21) Appl. No.: 17/274,664

(22) PCT Filed: Sep. 10, 2019
(86) PCT No.: PCT/SG2019/050452

§ 371 Date: Mar. 9, 2021

(30) Foreign Application Priority Data
Sep. 11, 2018 (SG) 10201807828T

(51) Int. Cl. A61G 7/057

(52) U.S. Cl. CPC A61G 7/05715; A61G 2203/70

(57) ABSTRACT

The present disclosure provides a device, which includes: a first layer provided with a waterproof, breathable pressure re-distribution medium and adapted for adhesion around an area of interest on a patient prognosed for occurrence of pressure ulcer; at least one second layer disposed under the first layer and provided with pores, adapted for reducing impact of friction and shear on the area of interest; at least one third layer disposed under the at least one second layer, and made of a hard material and adapted for providing support to the device; and at least one fourth layer disposed under the at least one third layer, provided with grooved features, which allow for redistribution of pressure applied on the device. The device, when placed on the area of interest, allows for redistribution of pressure, reduced shear and reduced friction on the area of interest, and may be used for prevention of pressure ulcers.

### TECHNICAL FIELD

The present disclosure relates generally to the field of patient care. In particular, the present disclosure relates to application of prophylactic measures to mitigate development of pressure ulcers in a patient.

### BACKGROUND

A pressure ulcer is a localised injury to the skin and the underlying tissue that results from pressure on the area, typically in combination with friction and shear. Pressure ulcers are commonly found in persons who are bedridden or are otherwise immovable. Pressure ulcers are found at areas on the body, which, due to immovability of the patient, has to bear constant pressure, friction and shear. Typically, pressure ulcers develop on skin covering bony areas of the body. Pressure ulcers are particularly risky for patients suffering from conditions such as from diabetes, cardiovascular conditions, incontinence and arthritis amongst others. In many such cases, the development of pressure ulcers can lead to complications such as gangrene, infection etc.

Currently, some preventive measures are being followed involving off-loading of pressure on areas of the body and repositioning patients such that any one area of the body is not constantly under pressure. Without being able to accurately judge the occurrence of pressure ulcers, such approaches have a typically low success rate.

Once a pressure ulcer is detected, recovery from the pressure ulcer is also paramount in order to reduce chances of infection, and to also minimise pain. Generally, a dressing is applied to the area to re-distribute pressure on the pressure ulcer. However, it is also imperative for the dressing to allow for removal of any moisture at the site of the pressure ulcer and allow for ventilation to the site of the ulcer. Further, it is preferable that the dressing be firmly adhered to the area of skin even when the patient moves, such that the area of skin is always protected. There is a need to regularly change dressing, which, when considering conventional dressing approaches followed, can be time consuming and painful to the patients.

United States patent document US20180008476 provides a dressing which includes a central portion and a border portion, and which further includes a backing layer, an adhesive body contact layer and a pad arranges in the central portion between the backing layer and the adhesive layer. The pad is symmetric about a central longitudinal line of the pad. The dressing can be used for prevention of pressure ulcers. However, the dressing of the cited patent document, with only one padded layer, may not provide adequate support to fully absorb and redistribute pressure across an area prognosed or diagnosed with a pressure ulcer.

Patent documents US20190192730, EP3498241 and CN103212105 provide dressings with single padded layers to support the pressure ulcer and to re-distribute pressure across the ulcer. This may not provide adequate support to fully absorb and redistribute pressure across an area prognosed or diagnosed with a pressure ulcer.

There is, therefore, a requirement in the art for a means to proactively and efficiently mitigate the development of pressure ulcers and, further, for a means to easily and safely treat the occurrence of the pressure ulcer.

### OBJECTS OF THE INVENTION

Another object of the present invention is to provide a device for applying prophylactic measure to an area of interest on the patient prognosed for occurrence of pressure ulcer.

Another object of the present invention is to provide a device for applying prophylactic measure that is safe and convenient to use.

Another object of the present invention is to provide a device for applying prophylactic measure that is economical.

### SUMMARY

The present disclosure relates generally to the field of patient care. In particular, the present disclosure relates to application of prophylactic measures to mitigate development of pressure ulcers in a patient.

In an aspect, the present disclosure provides a device suitable for applying prophylactic measure on an area of interest on a patient, which includes: a first layer provided with a pressure re-distribution medium and adapted for adhesion around the area of interest prognosed for occurrence of pressure ulcer in a patient; at least one second layer disposed under the first layer and provided with pores, wherein the at least one second layer is adapted for reducing impact of friction and shear on the area of interest; at least one third layer disposed under the at least one second layer, the at least one third layer made of a hard material and adapted for providing support to the prophylactic device; and at least one fourth layer disposed under the at least one third layer, the fourth layer provided with grooved features away from the direction of the area of interest, wherein the grooved features allow for redistribution of pressure applied on the device. The prophylactic device, when placed on the area of interest, allows for redistribution of pressure, reduces shear and reduced friction on the area of interest.

In a preferred embodiment, the first layer can have a length in the range of 5-36 cm and a breadth in the range of 2-31 cm such that the length and breadth facilitate encapsulation of the subsequent layers upon adhesion with the area of interest of the patient.

In a preferred embodiment, the prophylactic device can include a fifth layer disposed under the at least one fourth layer. The fifth layer can be provided with mesh designs adapted for passage of moisture and exudate from the area of interest.

In a preferred embodiment, the fifth layer is made of material selected from a group comprising silicone foam, polyurethane and/or a combination thereof.

In a preferred embodiment, the first layer is made of material selected from a group comprising breathable polyolefin-based materials such as polyethylene, polyamide, polyester, polyurethane, silicone and/or a combination thereof.

In a preferred embodiment, an adhesive is provided on the first layer to enable adhesion of the first layer on the area of interest, said adhesion being a soft adhesive such as silicone.

In a preferred embodiment, the at least one third layer is made of a hard material selected from a group comprising polyethylene, polyurethane and/or a combination thereof.

In a preferred embodiment, the at least one fourth layer can be adapted to be moisture absorbent and can be adapted to provide a cushioning effect.

In a preferred embodiment, the at least one fourth layer is made of material selected from a group comprising silicone foam, polyurethane and/or a combination thereof.

Various objects, features, aspects and advantages of the inventive subject matter will become more apparent from the following detailed description of preferred embodiments, along with the accompanying drawing figures in which like numerals represent like components.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

FIGS. 1A and 1B illustrate a schematic representation and an image respectively, of a device for applying prophylactic measures against pressure ulcers of a patient, in accordance with an embodiment of the present disclosure.

FIG. 2A illustrates schematic representations of exemplary mesh design on the “fifth layer”, in accordance with an embodiment of the present disclosure.

FIG. 2B illustrates schematic representations of tunnel designs on the “third layer”, in accordance with an embodiment of the present disclosure.

FIG. 2C illustrates schematic representations of the “third layer” with different tunnel designs, on application on the skin, in accordance with an embodiment of the present disclosure.

FIGS. 3A to 3E illustrate schematic representations of different configurations of a device for applying prophylactic measures against pressure ulcers of a patient, in accordance with an embodiment of the present disclosure.

### DETAILED DESCRIPTION

The following is a detailed description of embodiments of the disclosure depicted in the accompanying drawings. The embodiments are in such details as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Patients who are bed-ridden people, people with disabilities or otherwise immovable for extended periods of time are particularly susceptible to occurrences of pressure ulcers. A pressure ulcer is a localized injury to the skin and/or underlying tissue, typically over a bony prominence due to pressure on the skin and underlying tissue, where the pressure can block circulation of blood between the bony prominence and the region of skin and underlying tissue under pressure. The pressure ulcer can also occur due to additional shear and friction at the skin and underlying tissue. An important parameter to determine possibility of occurrence of a pressure ulcer at an area of skin is the pressure at the area, along with other secondary parameters, which can include environmental variables such as time of application, temperature and humidity and patient-specific data such as age and mobility level.

In an aspect, the present disclosure provides a device for applying prophylactic measure against pressure ulcers, which includes: a first layer provided with a pressure re-distribution medium and adapted for adhesion around the area of interest prognosed for occurrence of pressure ulcer in a patient; at least one second layer disposed under the first layer and provided with pores, wherein the at least one second layer is adapted for reducing impact of friction and shear on the area of interest; at least one third layer disposed under the at least one second layer, the at least one third layer made of a hard material and adapted for providing support to the prophylactic device; and at least one fourth layer disposed under the at least one third layer, the fourth layer provided with grooved features away from the direction of the area of interest, wherein the grooved features allow for redistribution of pressure applied on the device. The prophylactic device, when placed on the area of interest, allows for redistribution of pressure, reduced shear and reduced friction on the area of interest.

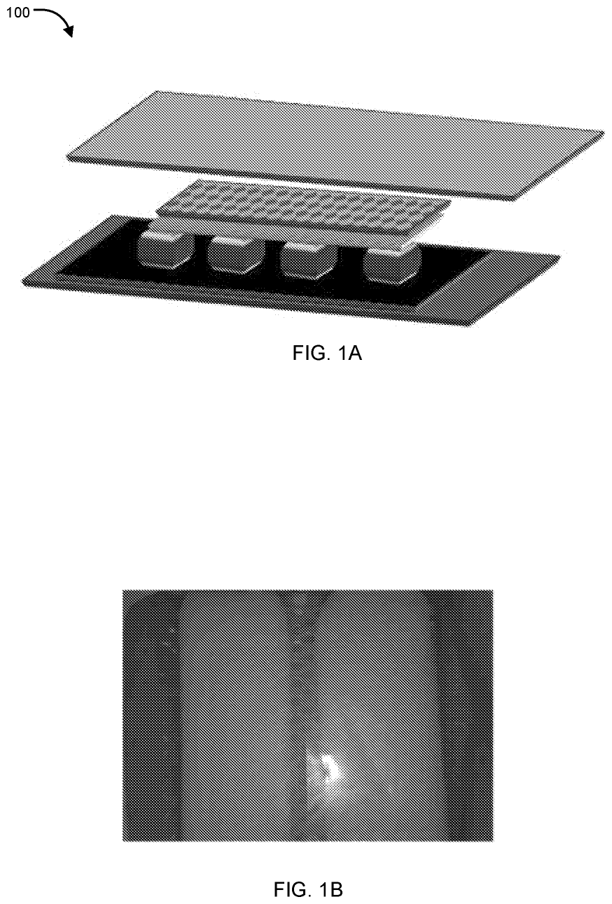

FIGS. 1A and 1B illustrate a schematic representation and an image respectively, of a device for applying prophylactic measures against pressure ulcers of a patient, in accordance with an embodiment of the present disclosure. The device 100 is a multi-layered dressing that can be applied on an area of skin of a patient.

One implementation of the device 100 is as a dressing to be applied in an area of skin of the patient where pressure ulcers have occurred or are predicted to occur, in order to provide relief to the patient by re-distributing pressure at the skin uniformly over a larger area. In the preferred embodiment, the device 100 can have two or more layers with a geometrical plane of symmetry. The device 100 is adapted to have characteristics such as, but not limited to uniform pressure re-distribution, shear reduction, friction reduction, moisture permeability and moisture absorption. The mentioned characteristics allow for delaying the onset of pressure ulcers and, in the even pressure ulcers have occurred, for mitigation of pain and quicker recovery. The device 100 can be adapted for easy application and removal, thereby allowing for easy, efficient and safe management of pressure ulcers. In the present embodiment, easy application and removal can be accomplished by having suitable soft adhesive on a layer of the device 100 adapted for adhesion with the skin. In the present embodiment, the layer can be designated as a “first layer”. The adhesives can be applied all along the perimeter or on two or more borders of the device 100 in such a way that the device 100 can adequately adhere to the skin around, but not on the pressure ulcer, thereby minimising pain and risk of damage to the wound. The adhesive used can be such that it does not irritate the skin, such as silicone gel. The “first layer” can be a thin film made of material such as, but not limited to breathable polyolefin-based materials such as polyethylene, polyamide, polyester, polyurethane and silicone. The planar dimensions of the “first layer” can depend on the area and location of application of the device 100 on the body.

In a preferred embodiment, the device 100 can include a “second layer” disposed under the “first layer”. The material of the “second layer” can be adapted to have perforations, or pores. The density and shape of the pores can vary, according to the location of application of the device 100 on the patient body. Further, the density of the pores and the shape of the pores can vary across the area of the device 100, according to the location of application of the device 100 on the patient body. The pores in the “second layer” facilitate a reduction in impact of friction and shear on the area of application due to its oscillatory motion between the “first layer” and subsequent layers. In the preferred embodiment, the pore sizes can vary between 0.5 nm to 100 nm.

In a preferred embodiment, the device 100 can include a “third layer” disposed under the “second layer”. The “third layer” can be between 1 mm and 3 mm thick and can be made of a hard material such as, but not limited to polyethylene and polyurethane. The “third layer” is adapted for managing the distribution of weight on the device 100 so as to redistribute the weight uniformly over the “first layer” and the “second layer”.

In a preferred embodiment, the device 100 can include a “fourth layer” disposed under the “third layer”. The “fourth layer” can be between 3 mm and 10 mm thick. The “fourth layer” can be made of a material such as, but not limited to polyurethane and silicone foam and can be adapted to have micro pores, which facilitates circular or oscillatory motion of the device 100 against the skin, thereby reducing the impact of friction and shear. Further, the material is also highly absorbent, such that any moisture at the at the site of the pressure ulcer can be absorbed. The “fourth layer” can be provided with tunnels in one or more directions, which can allow for better redistribution of pressure on the device 100. The tunnels can have different geometries and their width can vary between 2 mm and 30 mm.

In a preferred embodiment, the device 100 can have a “fifth layer” disposed under the “fourth layer”. The “fifth layer” is the layer in contact with the skin and can be flexible and have a mesh design of different shapes such as circle, square, hexagonal etc., and with mesh size diameters varying from 2 mm to 10 mm. The spaces allows for passing of exudate and moisture through the “fifth layer”. The “fifth layer” can be made of materials such as, but not limited to silicone, polyurethane and other similar materials.

In a preferred embodiment, the device 100 can have a “sixth layer”, which can be in contact with the “first layer”, thereby encapsulating the layers between. The “sixth layer” is adapted to preserve the adhesive on the “first layer”. The “sixth layer” can be removed before application of the device 100 on the area of skin. The “sixth layer” can be made of a material such as polyethylene.

In an exemplary embodiment, the length of the device can range between 10 and 36 cm and the breadth of the device can vary between 5 and 31 cm.

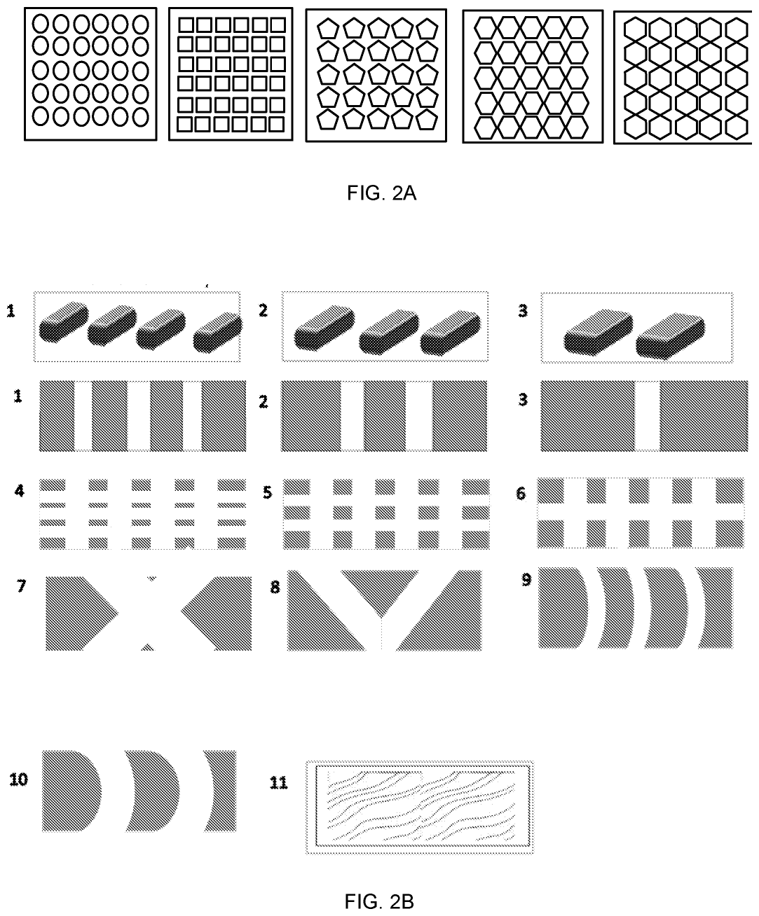

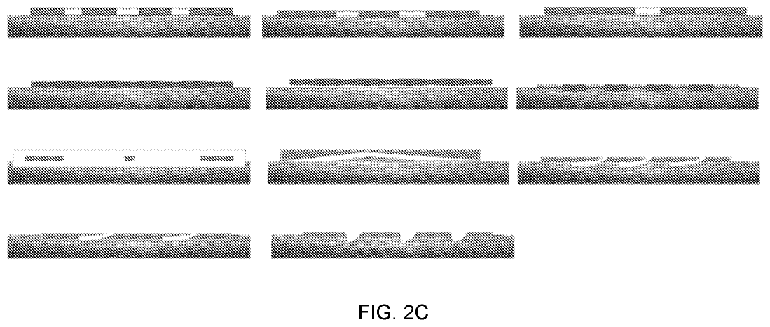

FIG. 2A illustrates schematic representations of exemplary mesh design on the “fifth layer”, in accordance with an embodiment of the present disclosure.

FIG. 2B illustrates schematic representations of tunnel designs on the “third layer”, in accordance with an embodiment of the present disclosure.

FIG. 2C illustrates schematic representations of the “third layer” with different tunnel designs, on application on the skin, in accordance with an embodiment of the present disclosure.

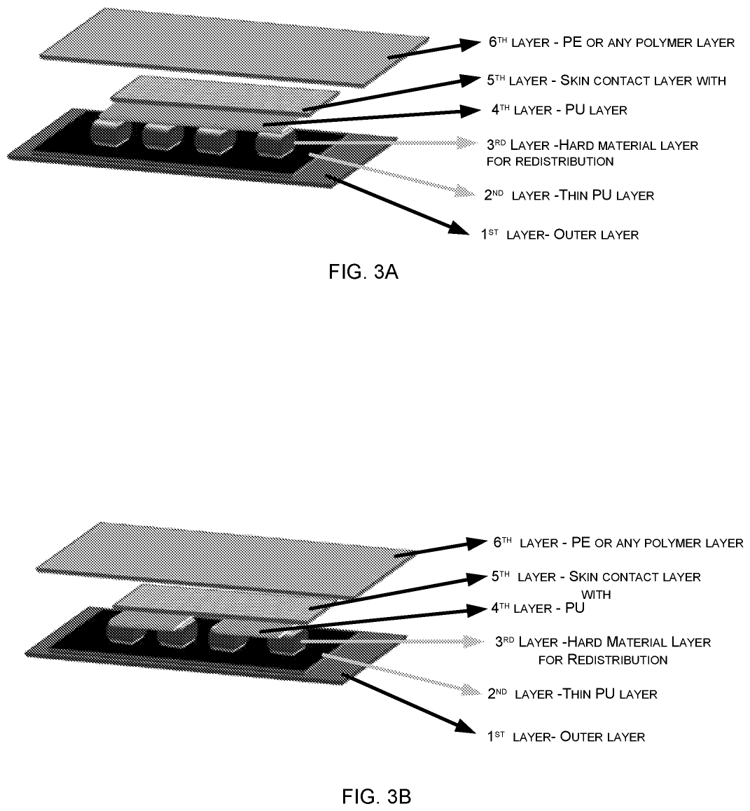

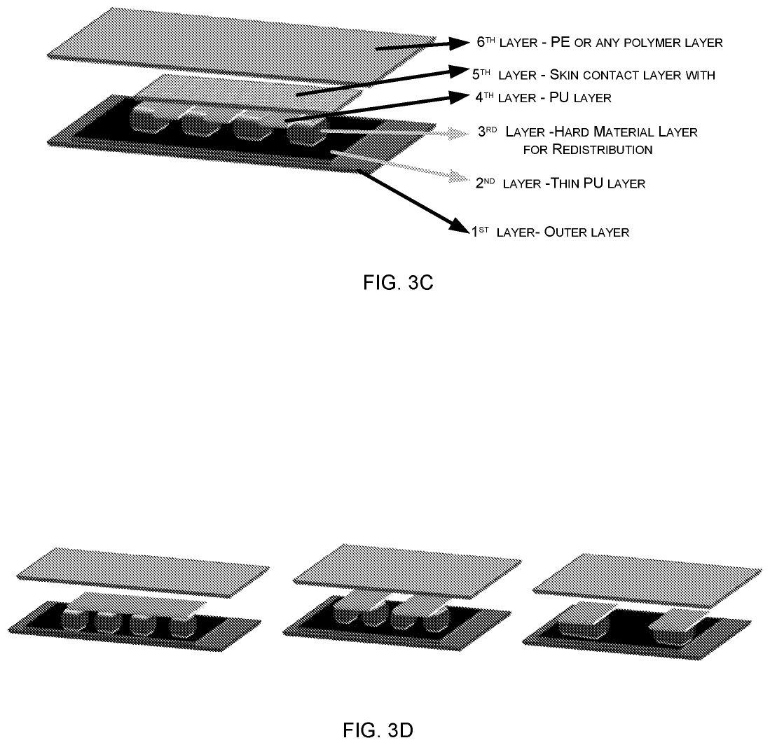

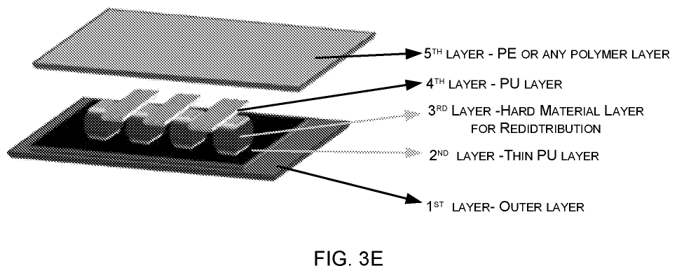

FIGS. 3A to 3E illustrate schematic representations of different configurations of a device for applying prophylactic measures against pressure ulcers of a patient, in accordance with an embodiment of the present disclosure. In different embodiments, the number of layers on the device 100 can be increased or decreased as required, based on area and location of application of the device 100 on the skin of the patient body.

Based on a study of the proposed device 100 for pressure ulcers, it can be demonstrated that the proposed device 100 can be effective in mitigation of development of stage-1 and stage-2 pressure ulcers.

Thus, aspects of the present disclosure provide a prophylactic device for applying dressing to the area of interest if an occurrence of pressure ulcer is predicted at the area of interest. The prophylactic device consists of multiple layers bonded together to provide reduced friction and shear as well as to provide uniform re-distribution of pressure on the area of interest. The prophylactic device is further configured to easy application and easy peel-off, thereby improving safety and efficiency of care. Application of the prophylactic device during early stages of pressure ulcer formation, or during detection of possibility of occurrence of pressure ulcers can greatly delay the onset of pressure ulcers in a patient.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The present invention provides a device for applying prophylactic measure to an area of interest on the patient prognosed for occurrence of pressure ulcer.

The present invention provides a device for applying prophylactic action that can effectively support and re-distribute pressure across a pressure ulcer.

The present invention provides a device for applying prophylactic action that can effectively support the pressure ulcer even during movement of the patient.

The present invention provides a device for applying prophylactic action that can absorb and/or wick moisture and any exudate from an area of pressure ulcer.

The present invention provides a device for applying prophylactic measure that is safe and convenient to use.

The present invention provides a device for applying prophylactic measure that is economical.

We claim:

1. A device suitable for applying prophylactic measure on an area of interest on a patient, said comprising:
a first layer provided with a waterproof, breathable pressure re-distribution medium and adapted for adhesion around the area of interest prognosed for occurrence of pressure ulcer in a patient;
at least one second layer disposed under the first layer and provided with pores, wherein the at least one second layer is adapted for reducing impact of friction and shear on the area of interest;
at least one third layer disposed under the at least one second layer, said at least one third layer made of a hard material and adapted for providing support to the prophylactic device; and
at least one fourth layer disposed under the at least one third layer, said fourth layer provided with grooved features away from the direction of the area of interest, wherein the grooved features allow for redistribution of pressure applied on the device, and
wherein the prophylactic device, when placed on the area of interest, allows for redistribution of pressure, reduced shear and reduced friction on the area of interest.

2. The device as claimed in claim 1, wherein the first layer has a length in the range of 5 to 36 cm and a breadth in the range of 2-31 cm.

3. The device as claimed in claim 1, wherein the prophylactic device comprises:
a fifth layer disposed under the at least one fourth layer, said fifth layer provided with mesh designs adapted for passage of moisture and exudate from the area of interest.

4. The device as claimed in claim 3, wherein the fifth layer is made of material selected from a group comprising silicone foam, polyurethane and/or a combination thereof.

5. The device as claimed in claim 1, wherein the first layer is made of material selected from a group comprising breathable polyolefin-based materials such as polyethylene, polyamide, polyester, polyurethane, silicone and/or a combination thereof.

6. The device as claimed in claim 1, wherein an adhesive is provided on the first layer, said adhesive being a soft adhesive such as silicone.

7. The device as claimed in claim 1, wherein the at least one third layer is made of a hard material selected from a group comprising polyethylene, polyurethane and/or a combination thereof.

8. The device as claimed in claim 1, wherein the at least one fourth layer is made of a material selected from a group comprising polyurethane, controlled/non-controlled pore sized polyurethane material or similar material and/or a combination thereof.

9. The device as claimed in claim 1, wherein the at least one fourth layer is made of material selected from a group comprising silicone foam, polyurethane and/or a combination thereof.
